(19) United States
(12) Patent Application Publication
(10) Pub. No.: US 2003/0130225 A1
(43) Pub. Date: Jul. 10, 2003

(54) Novel methods of treating local fungal and bacterial infections

(76) Inventors: Nawaz Ahmad, Monmouth Junction, NJ (US); Emilia C. Lonardo, Plainsboro, NJ (US); Kalpana J. Patel, West Windsor, NJ (US); Shun Y. Lin, Plainsboro, NJ (US); Lorraine L. Wearley, Westfield, NJ (US); J. Neal Matheson, Princeton, NJ (US); Brinda Wiita, Princeton, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/262,389

(22) Filed: Oct. 1, 2002

Related U.S. Application Data
(60) Provisional application No. 60/329,669, filed on Oct. 16, 2001.
(60) Provisional application No. 60/392,477, filed on Jun. 28, 2002.

(51) Int. Cl. A61K 31/7076; A61K 31/7072; A61K 31/496; A61K 31/4196; A61K 31/4178; A61K 31/522

(52) U.S. Cl. 514/45; 514/49; 514/263.38; 514/254.07; 514/397; 514/383

(57) ABSTRACT

This invention relates to novel compositions, methods of use, regimens and kits for treating local fungal and bacterial infections that are susceptible to treatment administered either locally or topically and systemically. The methods of this invention relate to ways in which to relieve symptoms of such infections in an unexpectedly shorter timespan than with conventional treatment with greater predictability than those methods currently known.

### BACKGROUND OF THE INVENTION

This invention relates to novel methods of treating local fungal and bacterial infections that are susceptible to treatment administered either locally or topically and systemically. The methods of this invention relate to ways in which to relieve symptoms of such infections in an unexpectedly shorter timespan than with conventional treatment.

Vaginal yeast infections are usually treated locally by vaginal application of creams, suppositories, and soft gelatin capsules, vaginal tablets and ointments containing antifungal agents. These products are not very convenient to use and have the undesired side effect of messiness associated with them. In recent years, an oral antifungal medication, fluconazole, the first of a new class of synthetic triazole antifungal agents, was approved for prescription and is available in tablet form for oral administration.

Consumers desire both convenience of use and freedom from the feeling of messiness associated with topically applied products.

Fluconazole is a systemic antifungal agent that is taken by mouth. It is fungistatic, which means it stops fungi from multiplying, but does not actually kill them. Thus, upon oral administration, it sometimes takes several days for symptoms to subside. While the symptoms linger, the patient does not have the perception of relief. The patient may also have an infection on the external skin or vulva along with the internal vaginal infection.

Oral fluconazole also has notable side effects such as headache, nausea, liver dysfunction, abdominal pain, skin rashes and in some cases diarrhea, dizziness, and potential birth defects. Furthermore, in its currently available prescribed dose, oral fluconazole cannot be taken in conjunction with a number of medications, including oral hypoglycemics, coumarin-type anticoagulants, cyclosporin, terfenadine, theophylline, phenytoin, rifampin, astemizole, rifabutin, and tacrolimus due to drug interaction.

In response to the lagging relief of symptoms concomitant with the use of oral fluconazole, some physicians have prescribed the use of topical creams, some containing miconazole nitrate or other ingredients to ease symptoms of the infections. External treatment options can range from external hydrocortisone cream to commercially available vaginal preparations containing azoles such as butaconazole, miconazole nitrate or tioconazole. However, combining oral fluconazole with other drugs, including azoles, can increase the potential for drug:drug interactions, which may have serious side-effects. Moreover, these external vulvar and vaginal products can also increase the potential for vulvar irritation and sensitization. In addition, topical and/or intravaginal preparations are available in a large variety of forms and concentrations. Should a patient purchase a high-concentration miconazole nitrate preparation intended only for intravaginal use and apply it to an infection-laden vulva, she may encounter a high degree of irritation and discomfort.

In addition, not all vaginal infections are caused by yeast. In fact, the most common vaginal infection is caused by bacteria, known as “bacterial vaginosis”. Current effective topical treatment of bacterial vaginosis is available only by prescription and utilizes 0.75% metronidazole as a vaginal gel once a day for five days or as a single two gram oral dose as a tablet. Another treatment for bacterial vaginosis utilizes the drug clindamycin. Metronidazole may also be administered as a seven-day course of treatment in 250 mg tablets taken once daily.

Localized fungal and/or bacterial infections have been treated via localized topical or oral systemic treatment with varying results. For example, onychomycosis (fungal infection of the nail) is treated with oral antifungals, such as itraconazole. Viral infections such as herpes simplex can also be treated by the topical or oral administration of antiviral medications. For instance, herpes labialis (viral infection of the lips) can be treated with oral or topical acyclovir.

Two separate clinical studies were conducted to evaluate the regimen on the efficacy of oral fluconazole 150 mg treatment for tinea corporis and tinea cruris as reviewed by Lesher, J. L. (J Am Acad Dermtol 1999;40:S31-4). They found that two doses over the course of two weeks were usually required for control of infections as the result of Epidermophyton floccosum and Candida, whereas three to four doses over the course of four weeks usually were required for other organisms.

Thus, although oral fluconazole therapy may be efficacious in treating various skin fungal infections, the drug must be administered for at least two to four weeks, or even eight weeks, to cure disease. Since most skin fungal infections are associated with a rash that is itchy, red and which evidences scaling or other unpleasant symptoms, faster cure and symptom relief are highly desirable.

Gupta A. K. and Shear, N. H. reviewed the newer oral antifungal agents used to treat onychomycosis over the last ten years, namely, itraconazole, terbinafine and fluconazole. Curing onychomycosis infections using such oral antifungal agents may take from four to eighteen months. Considering the long treatment duration and progressively worsening compliance usually associated with long treatment regimen of non-life-threatening diseases (such as onychomycosis), a shorter and yet efficacious oral antifungal therapy is certainly desirable.

Furthermore, currently approved oral antifungal agents may cause several types of adverse reactions, such as nausea, gastrointestinal distress, diarrhea, abdominal pain, cutaneous eruption, and central nervous system effects including headache and malaise. Although the newer antifungal agents are generally well tolerated with drug interactions that are usually predictable, close monitoring of hepatic functions are required due to the potentially grave consequences resulting from taking these medications (Physician's Desk Reference®, PDR® Electronic Library™, 2002). A shorter oral antifungal therapy would reduce the potential risk from the unwanted side effects such as liver toxicity and drug interactions.

Penlac™ antifungal nail lacquer is the first prescription topical therapy approved for the treatment of onychomycosis in toenails and fingernails in the U.S. It contains 8% ciclopirox, a broad-spectrum antifungal agent that inhibits the growth of dermatophytes in the nails. Penlac™ can only be used to treat mild to moderate form of onychomycosis, which does not involve fungal infection in the nail matrix (the “root” of the nail plate). Although Penlac™ is safe and convenient to use without systemic side effects, the treatment efficacy is low (i.e., less than 12%) and the treatment duration is lengthy (i.e., daily application for 48 weeks) (Physician's Desk Reference®, PDR® Electronic Library™ 2002). As with any prolonged treatment regimen, patient compliance is likely to be poor, which may further lower the treatment efficacy.

Mikami, Y. et al reported an increased level of activity in vitro using both miconazole and fluconazole at sub-MIC level on Candida albicans (Mycoses 35: 11-12, 321-7, Nov-Dec, 1992). They studied in vitro combination effect of miconazole and fluconazole against Candida albicans. When minimum (MIC) and sub-minimum (sub-MIC) inhibitory concentration and fractional inhibitory concentration (FIC) determinations were made, the combination was effective at concentrations well below their individual MICs (at sub-MIC levels). However, increased effect against Candida krusei was not confirmed. The authors did not discuss any details about how to use their findings, nor did they show whether fluconazole has any improved effects with other imidazoles and triazoles against Candida albicans, or against dermatophytes such as Trichophyton rubrum and Epidermophyton floccosum.

### SUMMARY OF THE INVENTION

Accordingly, the compositions, methods of use, regimens and kits of this invention provide predictable, minimally irritating means for treating local infections utilizing systemic and topical treatments. The compositions, methods of use, regimens and kits of this invention provide faster symptom relief to the patient and substantially eliminates the potential for drug:drug interactions, significantly decreases sensitization and irritation potential and provides a safe, effective and convenient product for patients and physicians.

The compositions and methods of this invention, therefore, relate to novel treatments of fungal and bacterial infections utilizing novel compositions, treatment regimens, and kits that enhance consumer preference and appeal.

In general, the compositions, methods and kits of this invention relate to the combined therapy of a patient suffering from a localized fungal or bacterial infection taking a systemic medication administered remotely from the site of infection and applying to the local situs of infection a novel topical composition that can serve to relieve the symptoms of the infection such as itch, pain, inflammation or the like.

Remote administration of antifungal or antibacterial medicament containing an antifungal or antibacterial active ingredient (or other pharmaceutically effective moiety) may be accomplished by any means known to one of ordinary skill in the art, including, but not limited to, oral, parenteral, transdermal, buccal, intramuscular, or intranasal. The medicament should be included in a composition containing a pharmaceutically-effective amount of the active ingredient in a pharmaceutically acceptable carrier. The medicament may be administered in one or more doses. If administered in more than one dose, the dosage level of active ingredient in the medicament may be the same in each dose, or may be first administered in a higher level or bolus and then in a lower maintenance dose.

In one embodiment of this invention, an oral antifungal medication is administered in conjunction with a topical antifungal medication in order to treat a fungal infection.

A series of imidazoles are approved for use as antifungal agents when applied vaginally. These antifungal products are available for over-the-counter use as creams, suppositories, vaginal tablets, soft gelatin capsules and ointments and for prescription use as creams and suppositories. These products have demonstrated safety and efficacy for treating vaginal fungal infections established by virtue of clinical studies required for marketing approval by the United States Food and Drug Administration as well as their long-term use after approval. These products not only vary in efficacy but in consumer preference and appeal.

In one preferred embodiment of the compositions, methods of use, regimens and kits of this invention, an oral antifungal medication, such as fluconazole, may be taken to treat a vaginal fungal infection at a currently approved dosage (about 150 mg single dose mg or greater) or at a lower dose (preferably, from about 75 to about 100 mg, but possibly less than about 125 mg). Concurrently and subsequently for up to about 7 days, the patient applies an external topical composition to her vulva from about one to about three times per day. More preferably, the external topical composition may be applied to the vulva less than about 5 days. Surprisingly, we have found that topical fluconazole-containing compositions are less irritating than many currently available topical antifungal compositions. We expect that concurrent topical and systemic administration of fluconazole may lead to faster speed to relief, faster speed to cure, faster and more complete cure as well as better lasting cure without recurrence than mere systemic administration of drug.

The kits of this invention preferably contain an oral medication packaged with an appropriately formulated topical composition that ameliorates symptoms of a local infection. More preferably, the kit should contain an oral medication and a topical composition containing an active ingredient for treating the local infection in a pharmaceutically acceptable carrier in amounts that are non-irritating to the local site of infection.

More preferably, in one embodiment of the kits of this invention, the kit should contain oral fluconazole and miconazole nitrate cream. Preferably, the oral fluconazole may be in present an amount of about 150 mg and the cream should contain about 2% miconazole nitrate by weight of the composition.

Most preferably, in one embodiment, the kit should contain oral fluconazole and topical cream containing from about 1 to about 3% fluconazole by weight of the composition.

The kits of this invention provide predictable and controllable doses of both oral and topical medicaments that provide both systemic and symptomatic relief from local infections without causing additional irritation tot the infected tissue. This affords a distinct advantage to the patient over current practice, in which a patient receives oral medication and, if directed, chooses a topical cream on his or her own. Self-selected topical preparations may be unpredictable in result, causing or potentiating drug:drug interactions or unnecessary additional irritation to already-inflamed and possibly abraded infected tissue.

The external topical compositions of this invention may contain an antifungal active agent, an antibacterial active agent, or both an antifungal and antibacterial agent, or a dual-action active ingredient or pharmaceutical agent. Acceptable antifungal agents are preferably chloroxylenol, undecyclenic acid, selenium sulfide, tolnaftate or iodochlorohydroxyquine or the like. Certain azole antifungal agents and their salts and esters may also be utilized. Preferably, imidazole antifungal agents may be utilized in the compositions and methods of this invention. More preferably, the antifungal agents may be selected from the following group: fluconazole, timidazole, secnidazole, miconazole nitrate, econazole, metronidazole, itraconazole, terconazole, posaconazole, ravuconazole, ketoconazole, clotrimazole, sapirconazole and the like, as well as their salts and esters.

Acceptable antibacterial agents are preferably metronidazole, timidazole, secnidazole, clindamycin, vaginal acidifying/buffering agents and the like. Such antifungal agents may assist in combating the infection locally. However, the external topical compositions of this invention would not be required to be applied internally in order to contact the internal situs of the infection, thus avoiding the unpleasant side effect of leakage and messiness engendered by current regimens.

External topical compositions of this invention may also contain an antiseptic agent, such as iodine, iodophors, chlorohexidine gluconate, thimerosal or hydrogen peroxide or the like. Such external topical compositions may assist in treating any secondary skin infections of the vulva.

External topical compositions of this invention may also contain skin protectants. By protecting the skin, not only does the composition soothe the site of infection; it also maintains the integrity of the skin to prevent additional damage and pain. Skin protectants may include allantoin, cocoa butter, dimethicone, kaolin, shark liver oil, petrolatum, vegetable oils, zinc oxide and others known to those of skill in the art.

Local anesthetics or antihistamines may also be employed in the external topical compositions of this invention in order to lessen the pain and itching caused by the local infection. Local anesthetics and antihistamines that are useful in the compositions of this invention include benzocaine, lidocaine, dibucaine, benzyl alcohol, camphor, resorcinol, menthol and diphenhydramine hydrochloride and the like.

Anti-inflammatories such as corticosteroids, including hydrocortisone acetate, may also be employed in the external topical compositions of this invention.

The external topical compositions of this invention may be in the form of emulsions such as creams, lotions, ointments, powders, microemulsions, liposomes or may be gels and liquids. Emulsions may include oil in water or water in oil emulsions. The external topical compositions of this invention may also include intravaginal dosage forms such as creams, ointments, gels, gelatin capsules, suppositories and the like.

Preferably, cream compositions of the present invention are oil in water (O/W) emulsions in which the oil phase is considered the internal or dispersed phase while the aqueous phase is considered the external or continuous phase. The oil phase of the compositions of this invention preferably contain cetyl alcohol, stearyl alcohol and isopropyl myristate. The aqueous phase preferably contains propylene glycol, potassium hydroxide and water.

Where an active ingredient, such as an antifungal compound, may be soluble in water, the ingredient is dissolved in the aqueous phase. Fluconazole, for example, is water-soluble and may be dissolved in the aqueous phase of the composition. In other cases, the active may not be water-soluble and therefore it is preferably uniformly dispersed and suspended throughout the cream after the cream is formed. For example, miconazole nitrate may be so dispersed. Benzoic acid or like preservatives may be used as preservatives to protect the topical preparation from bacterial growth.

In cream compositions according to this invention, a mixture of cetyl and stearyl alcohols, which act as auxiliary emulsifiers, impart to the oil phase of the compositions of this invention an HLB (hydrophilic lipophilic balance) value of about 15 and imparts to the cream the desired consistency and firmness. Preferably, the HLB value of emulsifiers used in the compositions of this invention is matched to that of the oil phase in order to achieve these attributes.

An ester, which acts as an emollient and lubricant is included in the composition. The ester is preferably a fatty acid ester and is selected from the group consisting of isopropyl stearate, isopropyl myristate, isopropyl palmitate and isopropyl laurate. Most preferably the ester is isopropyl myristate. The ester provides the cream with smoothness and lubricity for easy application and spreadability over the external vulvar area.

Preferably, propylene glycol is included as a humectant to prevent the cream from drying and formation of a crust due to moisture loss. The humectant is also used to solubilize the antifungal completely in the aqueous phase as in case of fluconazole or enhance the solubility of the antifungal as in case of miconazole nitrate. Humectant concentration used in the compositions of this invention also acts as an antimicrobial agent.

Polysorbate 60 is preferably used as a surfactant in the compositions of this invention as it has an established safety history, due to long-term use in vaginal compositions. This surfactant has an HLB value of about 15.0, which is very close to the HLB value of the oil phase of the cream embodiments of the compositions of this invention (15.2), thus it efficiently emulsifies the cream. Thus, polysorbate 60 efficiently emulsifies the cream and imparts a stable viscosity to the cream when used at a preferred concentration of about 3% to about 4%. Unlike commercially available creams, the compositions of this invention do not require the use of two separate surfactants. One surfactant alone, having an HLB of about 15 (which is close to the HLB value of the oil phase of the compositions) has been found to impart to the cream its novel viscous characteristic.

In preparing the vaginal creams of the invention, the following amounts by weight of the total composition are preferably used.

| Cetyl alcohol | 1% to 7% |
|---|---|
| Stearyl alcohol | 5% to 15% |
| Isopropyl myristate | 1% to 5% |
| Propylene glycol | 10% to 25% |
| Polysorbate 60 | 1% to 5% |
| Antifungal compound | 0.4% to 10% |
| Sodium or potassium hydroxide | sufficient to adjust |
| | pH between 3 to 7 |
| water | sufficient to make |
| | 100% |

The gel compositions of this invention are preferably aqueous gels where the antifungal is either completely dissolved in the gel vehicle as in the case of fluconazole or is suspended in the gel vehicle as in the case of miconazole nitrate.

The gelling agent, which is preferably a cellulose polymer is preferably selected from hydroxy- or carboxy-alkyl celluloses. More preferably, the gelling agent is selected from the group of carboxymethylcellulose, hydroxyethylcellulose, hydroxypropycellulose and hydroxypropylmethylcellulose, combinations thereof and the like. Most preferably, the gelling agent is sodium carboxymethylcellulose.

Polyhydric alcohols or polyols are used as humectants and plasticizers that are selected from polyhydric alcohols, including propylene glycol, glycerin or lower molecular weight polyethylene glycols such as polyethylene glycol 300 or polyethylene glycol 400 or combinations thereof and the like. More preferably, the polyols are propylene glycol and glycerin or a combination thereof.

The polyols used in the gel compositions of this invention act as plasticizers which serve to stabilize the gels and enhance their viscosity. These polyols or the combination thereof serve to solubilize the antifungal actives such as fluconazole. Thus, for example, fluconazole is in a relatively completely soluble state in the gel compositions of this invention. Polyols also act as humectants in order to retain the moisture within the compositions, preventing moisture loss and protecting the gels from drying and forming a crust on their surfaces.

Cellulose polymers may be used as gelling agents as they act as suspending agents, viscosity builders, thickeners and film formers. Additionally, cellulose polymers preferably used in the compositions of this inventions are hydrocolloids. Such hydrocolloids protect the vaginal mucous membranes and reduce irritation.

Lactic acid is preferably used to adjust the pH of the gel, although any known pH adjuster known to those of skill in the art may be utilized. The preferred pH is between about 3.5 and about 5.5. As the solubility of some antifungals such as fluconazole is pH-dependent, adjusting the pH to the appropriate level for the active ingredient is preferable in order to maximize the solubility of the active ingredient. The solubility of fluconazole, for example, is substantially maximized in this preferred pH range. This is also the preferred pH for vaginal applications, as the pH of a healthy vagina is between about 3.5 and about 5.

In preparing the vaginal gel compositions of the invention, the following amounts by weight of the total composition are preferably used.

| Propylene glycol | 15% to 25% |
|---|---|
| Glycerin | 5% to 15% |
| Cellulose gum | 1% to 4% |
| Antifungal compound | 0.25% to 2% |
| Lactic Acid | sufficient to adjust |
| | pH between 3 to 7 |
| water | sufficient to make |
| | 100% |

Lotion compositions of the invention utilize polyols such as propylene glycol and glycerin in combination with tocopherol acetate or vitamin E to solubilize antifungals intended for topical application in the external vaginal area. These lotion applications are novel in that the antifungals used are substantially completely dissolved, including miconazole nitrate, which is known to be very difficult to dissolve in various solvents known to the art. A distinct advantage of these soluble compositions is that very little azole antifungal compound is needed to give the desired relief. These applications have a lubricating and soothing perception to the tissues where they are applied such as external vaginal area.

In preparing the vulvar lotion compositions of the invention, the following amounts by weight of the total composition are preferably used.

| | |
|---|---|
| Propylene glycol | 35% to 50% |
| Glycerin | 45% to 55% |
| Tocopherol acetate | 0.25% to 1% |
| Antifungal compound | 0.25% to 2% |

Roll-on applications employ polyethylene glycols of desired melting point such as polyethylene glycol 1000, which has a melting range from about 37° C. to about 40° C., and polyethylene glycol 1450, which has a melting point from about 43° C. to about 46° C., to mold a roll-on which can be used to apply the antifungal or other active ingredient, which is completely dissolved in the polyethylene base. A fatty acid alcohol, preferably stearyl alcohol is added to afford stiffness and whiteness in order to improve the esthetic appearance. Vitamin E may be used to increase the emolliency of the roll-on preparation. The roll-on preparations of this invention are completely water soluble and thereby washable. Thus, these compositions are relatively free from messiness.

In preparing the roll-on compositions of the invention, the following amounts by weight of the total composition are preferably used.

| | |
|---|---|
| Polyethylene glycol 1000 | 90% to 95% |
| Polyethylene glycol 1450 | 3% to 7% |
| Tocopherol acetate | 0.5% to 1% |
| Antifungal compound | 0.25% to 2% |
| Stearyl alcohol | 0.25% to 1.5% |

The external topical compositions of this invention may be delivered by manual application. However, in order to avoid messy delivery and to keep the topical product in contact with the site of infections, it may also be applied by stick, swab, wipe, sanitary pad, pantyliner, wash, spray, roll-on, shampoo, depositing cleanser, solutions, or film-forming compositions, including nail lacquer or the like.

In one embodiment of the regimen of this invention, the patient would take a single oral dose of fluconazole in an amount of from about 25 to about 150 mg (although higher doses may be used, preferably from about 150 mg to about 250 mg) and concurrently apply an external topical cream containing from about 0.5% to about 5%, preferably 3% fluconazole or a gel containing from about 0.25% to about 5%, preferably 1.5% fluconazole. Such external topical composition may be applied 1 to 3 times per day for 1 to 7 days. We believe that application of such external topical cream in combination with a systemic orally-administered anti-infective would result in symptom relief that would be unexpectedly faster. Moreover, we believe that the dose of oral drug may be substantially lowered to about 100 mg, therefore decreasing the occurrence of side effects and drug interaction.

In another embodiment of the compositions and methods of this invention, an oral dose of antibacterial agent, such as metronidazole may be taken concurrently with application of an external topical composition containing one or more antibacterial agents to cure bacterial vaginosis. Alternatively, an oral dose of an antibacterial agent may be taken concurrently with application of an external topical composition containing an anti-inflammatory agent and/or a skin soothing agent or skin protectant. For example, oral administration of a systemic treatment could be accompanied by application of an azole-containing cream, lotion or gel from 1 to 3 times a day for between about 1 and about 7 days.

Alternatively, an oral dose of the antibacterial agent may be taken concurrently with application of an intravaginal buffering composition containing buffering agents, and optionally, an external topical composition preferably containing an anti-inflammatory agent and/or a skin soothing agent or skin protectant. The application of the intravaginal buffering composition surprisingly works with the systemically administered antibacterial agent to eliminate substantially the pathogenic bacteria, to minimize the symptoms of malodor and abnormal discharge, and to encourage the recolonization of beneficial bacteria in the vagina. Preferably, the applications of the intravaginal buffering compositions of this invention are continued for about three weeks after the systemic administration of the antibacterial agent, although such applications may be continued for a longer period of time.

In yet another embodiment of this invention, oral fluconazole may be administered to cure an occurrence of vulvovaginitis in conjunction with the application of an external topical composition containing an anti-inflammatory agent and/or a skin soothing agent or skin protectant.

In practice, the compositions of this invention may preferably be supplied as a kit containing both an oral dose of a medication and the external topical composition in a tube to be applied manually or an applicator, swab, sanitary napkin, pantyliner, stick, wipe or spray.

In another embodiment of this invention, oral medication, such as fluconazole or the like, may be taken to treat a vaginal fungal infection at a currently approved dosage (about 150 mg single dose mg or higher) or at a lower dose (preferably, from about 75 to about 100 mg, but more preferably less than about 125 mg). Concurrently and subsequently for up to about 7 days, the patient should apply daily a vaginal composition containing one or more than one anti-infection agent intravaginally (i.e., into her vagina). More preferably, the vaginal composition may be applied to the vulva for about 3 days, and most preferably, for 1 day. Concurrently applied with the oral fluconazole treatment, the intravaginal composition may be used with or without the use of the external topical composition for the vulvar tissues described earlier.

The anti-infection agent utilized in the compositions and methods of this invention may be selected from an antifungal, an anti-bacterial, an anti-viral, a probiotic agent or a combination thereof. The antifungal agent is preferably an azole, more preferably an imidazole, including but not limited to the following: itraconazole, fluconazole, voriconazole, terconazole, saperconazole, fenticonazole, sertaconazole, posaconazole, ketoconazole, miconazole, econazole, clotrimazole, bifonazole, butaconazole, tioconazole, oxiconazole, sulconazole, elubiol, isoconazole, flutrimazole and their pharmaceutically acceptable salts and the like. The antifungal agent may also be an allylamine or one from other chemical families, including but not limited to, terbinafine, naftifine, amorolfine, butenafine, ciclopirox, griseofulvin, undecylenic acid, haloprogin, tolnaftate, nystatin, iodine, rilopirox, BAY 108888, purpuromycin, and their pharmaceutically acceptable salts. The anti-bacterial agent may preferably include but is not limited to metronidazole, clindamycine, timidazole, ornidazole, secnidazole, refaximin, trospectomycin, purpuromycin, and their pharmaceutically acceptable salts or the like. The antiviral agent may preferably include but is not limited to immunomodulators, more preferably imiquimod, its derivatives, podofilox, podophyllin, interferon alpha, reticulos, cidofovir, nonoxynol-9, and their pharmaceutically acceptable salts. The probiotic is preferably probiotic organism, including but not limited to, Lactobacillus and Bifidobacterium species, preferably L. rhamnosus, L. acidophilus, L. fermentum, L. casei, L. reuteri, L. crispatus, L. plantarum, L. paracasei, L. jensenii, L. gasseri, L. cellobiosis, L. brevis, L. delbrueckii, L. helveticus, L. salivarius, L. collinoides, L. buchneri, L. rogosal, L. bifidum, B. bifidum, B. breve, B. adolescetis or B. longum or the like.

In another embodiment of this invention, oral medication such as fluconazole, may be taken to treat a skin fungal infection with an approved dose such as about 150 mg or at a lower dose. Concurrently and subsequently for up to about 7 days, the patient applies daily a topical composition containing one or more than one anti-infection agent, such as an antifungal agent, onto his or her skin.

Preferably, the oral dose to treat skin fungal infections is given weekly for four weeks, more preferably, for two weeks, and most preferably, is given once as a single dose treatment. The antifungal agent in the topical composition is preferably an imidazole, including miconazole, econazole, and ketoconazole. In a more preferred embodiment, oral fluconazole is used to treat a topical fungal infection in conjunction with a topical composition containing another imidazole antifungal compound. We believe that there would be a positive anti-infective action between fluconazole and the imidazole that would shorten the duration of the concurrent treatment of the skin fungal infections such as tinea pedis, tinea corporis, tinea cruris and tinea captis. Preferably, one oral fluconazole dose is administered and a topical antifungal composition for seven days or less, preferably three days.

The topical antifungal composition of this invention are also expected to provide fast relief of unpleasant symptoms such as itch, rash and scaling by rapidly killing off surface-bound fungi to prevent formation of skin-irritating toxins, while the oral fluconazole treatment ensures a rapid and substantially complete elimination of Infections located below superficial mucosal layers.

In another embodiment of this invention, oral medication, such as fluconazole or another effective systemic antifungal compound, may be taken to treat toenail or fingernail fungal infections (onychomycosis). An efficacious dose of fluconazole, which may be greater than but is preferably about 150 mg or at a lower dose (preferably, from about 75 to about 100 mg, but possibly less than about 125 mg) may be administered for one or multiple days for up to about 6 to 12 months. The patient preferably applies daily, or more preferably, once or twice per week, a topical composition containing one or more than one anti-infection agent, such as an antifungal agent, onto his or her fungus-infected nails and the surrounding skin. Preferably, the oral fluconazole dose to treat nail fungal infections is given weekly for about 12 weeks, more preferably, for about 8 weeks, and most preferably, for about 4 weeks. The antifungal agent in the topical composition is preferably an imidazole, including miconazole, econazole and ketoconazole. The surprisingly, the combination of fluconazole and imidazole enables a shorter duration of the concurrent treatment of the nail fungal infections than would have been expected. The antifungal agent from the topical composition penetrates into the nail plate and into the underneath nail bed, while the oral fluconazole migrates from blood circulation into the nail matrix and nail bed, and into the nail plate to exert the antifungal action to substantially completely eliminate the fungal pathogens in the nail and surrounding skin tissues.

This combination therapy should provide better efficacy and shorter treatment duration than the currently available topical onychomycosis product, and should require less oral fluconazole than current oral therapy, hence reducing any potential side effects of the drug. In other embodiments, other approved antifungal compounds may be administered orally in a similar regimen in conjunction with topical antifungal treatment in order to achieve shorter duration treatments, lower probability of side effects and better patient compliance.

The topical antifungal composition in the present invention to treat skin and nail fungal infections may be in any pharmaceutically acceptable dosage forms, including but are not limited to, cream, lotion, solution, spray, aerosol, powder, ointment, gel, film-forming formulation, depositing formulation, nail lacquer, rinsing formulation, shampoo, and conditioner.

The following examples of the compositions, regimens and methods of this invention serve to illustrate but not to limit the scope of this invention.

### EXAMPLE 1

### Examples of External Topical Creams

A: Antifungal Cream: An antifungal cream according to the invention may be made using the following ingredients using the processes set forth above and those known to individuals of ordinary skill in the art:

| Ingredient | Weight/Weight % |
|---|---|
| Cetyl Alcohol | 3.00 |
| Stearyl Alcohol | 8.50 |
| Isopropyl Myristate | 1.00 |
| Propylene glycol | 20.00 |
| Polysorbate 60 | 3.00 |
| Imidazole antifungal | 0.25 to 2.00 |
| (preferably fluconazole | |
| or miconazole nitrate) | |
| Benzoic Acid | 0.10 to 0.50 |
| Potassium hydroxide or | to adjust the pH between |
| Lactic Acid | 3.5 t0 5.5 |
| Water | QS to 100% |

B. Antibacterial Compositions: Antibacterial compositions according to this invention may be made using the following ingredients:

| Ingredient | Weight/Weight % |
|---|---|
| Cetyl Alcohol | 3 |
| Stearyl Alcohol | 8.5 |
| Isopropyl Myristate | 1 |
| Propylene glycol | 20 |
| Polysorbate 60 | 3 |
| Antibacterial | 0.25 t0 2 |
| | (metronidazole) |
| Benzoic Acid | 0.1 to 0.5 |
| Potassium hydroxide or | to adjust the pH between |
| Lactic Acid | about 3.5 and about 5.5 |
| Water | QS to 100% |

C. Skin-soothing Composition: A skin-soothing composition may be made in accordance with this invention using the following ingredients:

| Ingredients | Weight/Weight % |
|---|---|
| Cetyl Alcohol | 3 |
| Stearyl Alcohol | 8.5 |
| Isopropyl Myristate | 1 |
| Propylene glycol | 20 |
| Polysorbate 60 | 3 |
| Soothing Agent | about 0.25 to about 2 |
| (for example, shark liver oil) | |
| Benzoic Acid | 0.1 to 0.5 |
| Potassium hydroxide or | to adjust the pH between |
| Lactic Acid | 3.5 t0 5.5 |
| Water | QS to 100% |

D. Gel composition:

A gel composition for external topical application in accordance with this invention may be made using the following ingredients:

| Ingredients | Weight/Weight % |
|---|---|
| Fluconazole | 1 |
| Propylene Glycol | 20 |
| Glycerin | 10 |
| Cellulose Gum | 2 |
| Lactic Acid | (to adjust the pH |
| | between 3.5 to 5.5) |
| Purified Water | QS to 100% |

E. Lotion Composition:

A lotion composition for external topical application in accordance with this invention may be made using the following ingredients:

| Ingredients | Weight/Weight % |
|---|---|
| Fluconazole or miconazole nitrate | 0.25 |
| Propylene Glycol | 44.25 |
| Glycerin | 50 |
| Vitamin E | 0.5 |
| Total | 100.00% |

F. Roll-on composition:

A roll-on composition for external topical application may be made using the following ingredients:

| Ingredients | Weight/Weight % |
|---|---|
| Fluconazole or miconazole nitrate | 0.25 |
| Polyethylene Glycol 1000 | 94.00 |
| Polyethylene Glycol 1450 | 4.00 |
| Vitamin E | 0.25 |
| Stearyl Alcohol | 0.50 |
| Total | 100.00% |

### EXAMPLE 2

### Lower Dose of Oral Antifungal Active Required when Used in Conjunction with External Topical Composition Containing an Antifungal Active

Oral doses composed of (1) about 100 mg or (2) about 75 mg of fluconazole may be administered in a single oral dose in conjunction with an external cream, gel or lotion containing fluconazole as set forth above in Example 1A, 1D or 1E applied about 1 to about 3 times a day for from 1 to about 7 days.

The novel combination regimen of oral and topical fluconazole will be investigated in double-blind, randomized, parallel-group clinical trials evaluating the efficacy of cure of vaginal yeast infections and the speed and completeness of vulvovaginal symptom relief assessed by patients. Patients will be instructed to take one oral table on the day of the baseline visit. After screening procedures are completed, and to apply the topical preparation to the vulva from about 2 to about 3 times daily for up to seven days after taking the single oral dose. Therapeutic, mycological, and clinical cure rates will be assessed at a return visit scheduled 21-30 days later.

### EXAMPLE 3

### Faster Perception of Symptomatic Relief (Clinical Cure) When Vaginal Antifungal is Used in Conjunction with External Vulvar Topical Composition

A combination regimen of oral fluconazole and topical vulvar compositions containing no antifungal drugs will be investigated in double-blind, randomized parallel-group clinical trials evaluating the efficacy of cure of vaginal yeast infections and the speed and completeness of vulvovaginal symptom relief assessed by patients.

Patients will be instructed to take one oral tablet on the day of the baseline visit, after screening procedures are completed, and to apply the topical preparation to the vulva about 2 to about 3 times daily for up to 7 days after taking the single oral dose. After administering the oral tablet and applying the topical preparation, patients will record their vulvovaginal symptoms every half-hour for 3 hours (30 min, 60 min, 90 min, 120 min, 150 min, and 180 min). Subsequently, on days 2 through 7, patients will record their symptoms and dates and times of cream application. The time when symptoms were initially partially relieved and the time when symptoms were completely relieved will be recorded.

### EXAMPLE 4

### Novel Delivery Forms of External Topical Compositions

A. Applicator Stick or Swab

A stick with a cotton tip applicator impregnated with Cream (Example 1A), Gel (Example 1D) or Lotion Example E) may be used to apply the formulations 1A, 1D or 1E for about 1 to about 3 times a day for 1 to 7 days.

B. Wipe

A 1.5″×1.5″ swab constructed of a soft nonwoven or woven fabric, preferably a blend of cotton and rayon fibers impregnated with Cream (Example A), Gel (Example D) or Lotion Example E) applied 1 to 3 times a day for 1 to 7 days.

C. Sanitary Pad or Pantyliner

A sanitary pad or pantyliner may be impregnated with Cream (Example 1A), Gel (Example 1D) or Lotion Example 1E) applied 1 to 3 times a day for 1 to 7 days.

D. Spray

A Lotion (Example 1E) to be applied as a spray via a pump or aerosol 1 to 3 times a day for 1 to 7 days.

The aerosol spray will consist of the following component parts:

Propellant

Container

valve and actuator

product concentrate

The product concentrate will contain antifungal in a soluble state as a clear solution of Example 1E.

The preferred propellant is hydrocarbon propellant selected from butane, isobutane or propane or a combination thereof.

Containers may be tinplate, aluminum, stainless steel or glass that must withstand pressure between 140 to 180 psig at 130° F.

The valve used will be constructed out of the materials approved by Food and Drug Administration for pharmaceutical aerosols.

The actuator will be such that will deliver or and direct the spray in the proper and desired form and allow easy opening and closing of the valve.

E. Roll-On

Example 1F may be applied to a reservoir in contact with a roller-ball that can be used 1 to 3 times a day for 1 to 7 days.

### EXAMPLE 5

### Oral Administration of Fluconazole and External Application of Miconazole-Nitrate Containing Composition with One Application of Cream

This combination regimen will consist of a single-dose oral fluconazole and an external vulvar cream containing 1-4% miconazole nitrate.

The cream is the same miconazole nitrate formulation as commercially available in Monistat®-7 or Monistat®-3 (Personal Products Company, Skillman, N.J.) and applied externally once, twice or three times daily for 1 to 7 days.

### EXAMPLE 6

### In vitro Permeation of Fluconazole in Human Skin

6A. In vitro Permeation of Fluconazole from 1% Fluconazole Gel and 3% Fluconazole Cream Through the Human Vaginal Mucosal Membrane

The objective of this experiment was to assess the permeation of fluconazole from either 1% fluconazole gel (Example 1D) or 3% fluconazole cream (Example 1A) through the human vaginal mucosal membrane. Full-thickness human vaginal mucosal membrane was obtained from the National Disease Research Interchange (Philadelphia, Pa.) or the Cooperative Human Tissue Network (Philadelphia, Pa.).

The vaginal mucosal membrane was cut and mounted onto 5-mm diameter Franz diffusion cells (n=3). Normal saline solution (also containing 0.01% of an antibacterial agent) was used as the receptor fluid. The receptor fluid was mixed with a magnetic stirring bar and maintained at 37° C. by a circulating water bath, which circulated water through the water jacket of the diffusion cells. Integrity of the human vaginal mucosal membranes was evaluated with ³H-water before use.

At the beginning of the study, a dose of approximately 100 micro-liters of the fluconazole gel or cream was applied to the mucosal membrane surface using a syringe. The donor cells were then covered with Parafilm after dosing to provide occlusion in order to mimic in-use conditions. The receptor fluid was sampled (300 μL) periodically up to 72 hours post-dose. After each sampling, the diffusion cells were replenished with fresh receptor fluid to the recorded volume. Test material in the receptor fluid was analyzed by High performance Liquid Chromatography (HPLC) for fluconazole content. The flux (steady state permeation rate) of fluconazole permeation through the human vaginal mucosal membrane for each formulation was determined based on the result of this in vitro permeation experiment.

Both fluconazole gel and cream resulted in a flux of 0.8±0.4 μg/cm²/hour.

6B. In vitro Permeation of Fluconazole from Aqueous Solutions Containing Fluconazole Through the Human Cadaver Skin

Three aqueous solutions were used in this experiment: fluconazole-saturated saline solution, fluconazole-saturated 45% aqueous propylene glycol solution, and fluconazole-saturated polyethylene glycol (PEG) 400 solution. The respective fluconazole concentration in each solution was determined. A specimen of the human cadaver skin (female, 45 years old, abdominal) was obtained the National Disease Research Interchange in Philadelphia, and was dermatomed to 350 μm using a Padgett Dermatome (Kansas, MO). The experimental procedure was similar to that described in the Example 6A. The results are shown in the following:

| Composition of | Fluconazole | Fluconazole | |
|---|---|---|---|
| Aqueous Solution | Fluconazole | Flux (ng/cm²/hr) | |
| Saline | 0.5% | 7 ± 2 | (n = 3) |
| 45% propylene glycol | 1.7% | 5 | (n = 2) |
| 45% polyethylene glycol | 1.5% | 3 | (n = 2) |

The permeation results from the Examples 6A & 6B demonstrate that fluconazole permeation rates into and through the human vaginal mucosa and skin are extremely low, although though the former was approximately 100-200 fold higher than the latter. The surprising finding of low skin and vaginal mucosal permeabilities of fluconazole has practical significance: it suggests that, after topical application of an external fluconazole cream or gel to the vulvovaginal region, the amount of fluconazole that could be absorbed into the body would be insignificant. Thus, topically applied fluconazole would not be eliminated from the target site by the absorption process. The data also confirmed that, if fluconazole were administrated to patients only by oral tablets, the amount of fluconazole that could reach the external vulvovaginal region by diffusion through the skin and mucosal barriers would be very low. This may explain the clinical findings that, while the time to reach peak plasma concentration of fluconazole after oral dosing was only about 3 hours (Debruyne D, “Clinical pharmacokinetics of fluconazole in superficial and systemic mycosis”, Clinic. Pharmacokinet. 33:1, pages 52-77, Jul, 1997), the average time for the onset of symptom relief is about 2.4 days (Slavin, M B et al., “Single dose oral fluconazole vs. intravaginal terconazole in treatment of Candida vaginitis, Comparison and pilot study”, J. Fla. Med. Assoc., 79:10, pages 693-696, October 1992). It has been reported that fluconazole, after a single dose of oral administration, reaches vaginal tissue and vaginal fluid in 2-3 days (2000 Physician's Desk Reference, Medical Economics Company, Inc., Montvale, N.J., pages 2338-2342).

Thus, topical application of an external cream or gel containing fluconazole would not only be expected to, surprisingly, significantly reduce the onset time of symptom relief among Vulvovaginal Candidiasis patients, but also would have a prolonged therapeutic action. It also suggests that with the topical treatment by a fluconazole composition, the dose of oral fluconazole can be substantially reduced, since the rapid build-up of a high drug concentration in the local tissues has already been achieved.

### EXAMPLE 7

In vitro Comparative Evaluation of Tissue Irritation by Topical Fluconazole Gel and Cream vs. a Commercial Miconazole Nitrate Cream

Potential dermal irritation is often evaluated using an in vitro microsomal enzyme reduction method described by Beridge, M V, et al., (“The biochemical and cellular basis of cell proliferation assays that use tetrazolium salts, Biochemica”, 4, pages 14-19, 1996). The following commercial bioassay kit is available based on this principle: the EpiDerm™ Skin Model Bioassay Kit (MatTek Corporation, Ashland, Mass.). Simply put, the bioassay determines the toxicity of a test composition by placing it on the surface of a cultured human epidermis cell membrane, and the percentage of epidermal cell death caused by contacting the test composition over time provides a quantitative measure of potential dermal irritation for the test composition.

The aforementioned bioassay was conducted using the EpiDerm™ Skin Model Bioassay Kit with the standard experimental procedure on 1% fluconazole gel (Example 1D), 3% fluconazole cream (Example 1A), and a commercial external symptom (itch) relief cream containing 2% miconazole nitrate (Monistat external vulvar cream, Advanced Care Products, Ortho Pharmaceutical Corp., Raritan, N.J.).

The test results are summarized as follows,

| Test Composition | % of Viable Cells at 24 hours |
|---|---|
| 1% fluconazole gel | 80% |
| 3% fluconazole cream | 97% |
| 2% miconazole nitrate cream | 60% |

Surprisingly, the fluconazole gel had a significantly higher percentage of epidermal cells remaining viable at 24 hours in comparison to a commercial itch relief cream. The comparison was even more striking and surprising when 3% fluconazole and 2% miconazole nitrate creams were compared. This result indicates that both fluconazole compositions were much less irritating and milder than the commercial itch relief cream. This property of an external itch relief composition is extremely important to the patients who are already suffering from the skin irritation symptom from pathogenic fungal toxins.

### EXAMPLE 8

### Preclinical Data

8A. In Vitro Data

A preclinical in vitro EpiDerm™ assay was conducted on 3% Fluconazole Cream and 1% Fluconazole Gel. The EpiDerm™ Skin Model Bioassay Kit was used to assess the potential dermal irritation of the test materials. The MTT conversion assay was used to assess cellular metabolism after exposure to the test article after various exposure times resulting in ET₅₀, the duration of exposure resulting in a 50% decrease in MTT conversion in test article treated EpiDerm™ cultures, relative to control. The ET₅₀ value for 3% Fluconazole Cream was determined to be greater than 24 hours with a 97.1% cell viability. The ET₅₀ values for 1% Fluconazole Gel was determined to be greater than 24 hours with the percent cell viability at 24 hour exposure of 79.7%. Therefore, the 3% Fluconazole Cream and the 1% Fluconazole Gel formulations of this invention are predicted to be non-irritating to the vaginal epithelium.

In vitro percutaneous absorption experiments using human vaginal tissue sections were performed separately with fluconazole at 3% in a cream formulation and at 1% in a gel formulation under infinite dose conditions. The experiments were carried out over 72 hours and the concentration of fluconazole in the diffusion cell receptor fluids were determined by HPLC. The experimentally measured dermal flux (J) of 3% fluconazole cream formulation was 0.8±0.4 μg/cm²/hour (n=3; 95% C.I.) and the dermal flux of 1% fluconazole gel formulation was 0.8±0.4 μg/cm²/hour (n=3; 95% C.I.)

8B. In Vivo Toxicity Data

A 10-day rabbit vaginal irritation assay was conducted to determine the irritation potential of 3% Fluconazole Cream and 1% fluconazole gel on the vaginal epithelium. In this study, 1 ml per dose of 3% Fluconazole Cream, or 1% Fluconazole Gel were each administered vaginally to their respective group of 10 rabbits daily for 10 consecutive days. Additionally, 1 ml per dose of Fluconazole Cream Placebo, or Fluconazole Gel Placebo, was each administered vaginally to its respective group of 6 rabbits daily for 10 consecutive days. A sham control of 6 animals was included in this study. All animals were sacrificed on day 10. Vaginal administration of 3% Fluconazole Cream, 1% Fluconazole Gel, and their respective placebo formulations to rabbits daily did not cause any significant pharmacotoxic effects or adverse effect on appearance, behavior, or body weight gain of test animals. No significant adverse effects were noted during gross necropsy. Vaginal tissues were fixed and submitted for histological evaluation. Tissues were evaluated and scored for epithelium, leukocyte infiltration, vascular congestion and edema. The vaginal administration of 3% Fluconazole Cream, Fluconazole Cream Placebo, 1% Fluconazole Gel, and Fluconazole Gel Placebo to rabbits for 10 consecutive days caused minimal irritation to the vagina with an average composite scores of 2.7, 3.4, 1.7 and 1.8, respectively, to the vaginal epithelium. The average composite scores of the test articles and placebo formulations are similar to the sham control group composite score of 2.2. The total mean severity scores were less than 11.5, and the vaginal responses to 3% Fluconazole Cream, 1% Fluconazole Gel, and their respective placebo formulations were graded as acceptable. The 3% Fluconazole Cream, 1% Fluconazole Gel, and their respective placebo formulations are minimal irritants to rabbit vaginal epithelium.

A 3-day rabbit penile irritation study was conducted. In this study, 0.2 ml per dose of Fluconazole 3% Cream, Fluconazole 1% Gel, and their respective placebo formulations, were administered by direct application to the penis of each animal in each group for 4 hours daily for 3 consecutive days. Five groups of male New Zealand White rabbits were evaluated (21 rabbits total). Three rabbits served as a sham control receiving 0.9% saline. Six rabbits in each test group received either 3% Fluconazole Cream or 1% Fluconazole Gel. Three rabbits in each placebo group received either the placebo for the 3% Fluconazole Cream or placebo for the 1% Fluconazole Gel. Test sites for all animals were subsequently examined and scored for signs of erythema and edema prior to dosing and Days 1 and 2 and approximately 24 hours and 48 hours (Day 3 and Day 4) after the final treatment. Macroscopically, little irritation (transient, slight erythema) was produced in all of the groups. Microscopically, several lesions were observed in the sham control group, the 3% Fluconazole Cream group, and the Fluconazole Gel Placebo group. These lesions normally occur and are not test article related. Chronic, active inflammation was observed in the Fluconazole Cream Placebo group, the 1% Fluconazole Gel group, and the Fluconazole Gel Placebo groups. This inflammation was considered to be minimal. The 1% Fluconazole Gel group had the most lesions overall but inflammation was still considered to be minimal.

Observations made during the in-life portion of this study, as well as gross necropsy observations indicate there were no significant findings for 3% Fluconazole Cream, 1% Fluconazole Gel, or their placebo formulations. The group mean irritation scores for all groups were similar to the group mean irritation score for the sham control. The 3% Fluconazole Cream, the 1% Fluconazole Gel, and their respective placebos are considered to be nonirritating to the rabbit penile epithelium.

The dermal sensitization potential of 3% Fluconazole Cream and 1% Fluconazole Gel was evaluated in Hartley albino guinea pigs. Five male and five female guinea pigs were topically treated with 3% Fluconazole Cream once a week for 3 consecutive weeks. Five male and five female guinea pigs were topically treated with 1% Fluconazole Gel. Following a 2 week rest period, a challenge was performed whereby each group of the 20 test and 10 previously untreated (naive) challenge control guinea pigs were topically treated with 3% Fluconazole Cream or 1% Fluconazole Gel. Challenge responses in the test animals were compared with those of the challenge control animals. Following challenge with 3% Fluconazole Cream and 1% Fluconazole Gel, dermal scores of 0 were noted in all the test and challenge control animals at the 24 and 48 hour scoring intervals. Therefore, group mean dermal scores were noted to be 0.0 in the test and challenge control animals for both 3% Fluconazole Cream and 1% Fluconazole Gel. Under the conditions of this study, 3% Fluconazole Cream and 1% Fluconazole Gel are not considered to be contact sensitizing agents in albino guinea pigs.

### EXAMPLE 9

### In vivo Administration of Oral Fluconazole in Conjunction with Topical Fluconazole

Thirty-five female albino rabbits vaginally infected with Candida albicans were randomly assigned to seven dosage groups, 5 rabbits per group. One group of vaginally infected rabbits was untreated rabbits were treated once (day 1 of study) with oral doses of 0.5, 1.0 or 2.0 mg/kg of fluconazole either alone or in combination with 50 ul of 3% fluconazole cream applied perivaginally. By 72 hours post dosage, 40%, 100%, and 60% of the rabbits in the 0.5, 1.0, and 2.0 mg/kg fluconazole dosage groups, respectively, had no Candida albicans identified in a culture of the vaginal mucosa as compared to 20% in the untreated group. Rabbits administered oral fluconazole in combination with perivaginally applied 3% fluconazole cream had 100%, 100%, and 80% negative vaginal culture results 72 hours after administration of 0.5, 1.0, and 2.0 mg/kg doses of oral fluconazole. Minimal irritation to the vaginal epithelium was seen in the groups receiving oral fluconazole with 3% fluconazole external cream as compared with mild irritation seen in the oral fluconazole group. Histologically, leukocyte infiltration in the vaginal epithelium was less in the groups receiving the external cream. This demonstrates that there is a reduction in symptoms when the 3% fluconazole external cream is used in combination with an oral dose. Serum levels of fluconazole were the same in both treatments. Therefore, orally administered fluconazole in combination with 3% fluconazole external vaginal cream is more efficacious than orally administered fluconazole alone in treating vaginal candidiasis, less irritating to the vaginal epithelium, and does not increase the systemic exposure of fluconazole.

### EXAMPLE 10

### Oral Administration of Fluconazole in Combination with Topical Miconazole Nitrate or Topical Fluconazole

Twenty-one female (Hra:NZW) SPF rabbits inoculated with Candida albicans were randomly assigned to four dosage groups, three rabbits in Group 1 and six rabbits per dosage group in Groups 2 through 4. Rabbits assigned to Group 1 were untreated. Rabbits assigned to Groups 2 through 4 were administered 3 mg/kg fluconazole orally once on day 1. Fluconazole gel (1%), fluconazole cream (3%) or miconazole nitrate cream (2%), respectively was applied perivaginally for three days (days 1, 2 and 3 of study) at a dose volume of 50 μl per rabbit. There were no adverse clinical observations related to the test articles. Body weights, body weight changes and feed consumption values were unaffected by the test articles. The vaginal irritation scores were graded as minimal for each group with composite scores of 3, 2, 3 and 4 for Groups 1 through 4 respectively. The scores suggest a less irritating trend for fluconazole cream and gel as compared to miconazole nitrate 2% cream.

### EXAMPLE 11

### Oral Administration of Fluconazole and External Application of Fluconazole Containing Composition with Application of Cream or Gel

This combination regimen will consist of a single-dose oral fluconazole and an external topical cream containing 1-3% fluconazole for the treatment of athelete's foot or another topical fungal infection, such as tinea corporis, tinea cruris or tinea captis.

The topical external application of fluconazole may be selected from Example 1 and should be applied externally once or twice daily for 3 to 5 days.

What is claimed is:

1. A method of treating a local fungal or bacterial infection comprising oral administration of an effective amount of anti-infective agent and concurrent local administration of an external topical composition comprising an effective amount of a topical relief agent to ameliorate local symptoms.

2. A method of treating a local fungal infection comprising oral administration of an antifungal-effective amount of an antifungal agent and concurrent local administration of an external topical composition comprising an antifungal agent

3. A method of treating a vaginal fungal infection comprising oral administration of an antifungal-effective amount of an antifungal agent and concurrent vulvar administration of an external topical composition comprising an antifungal agent.

4. A method of treating a vaginal fungal infection comprising oral administration of fluconazole and concurrent vulvar administration of an external topical composition comprising fluconazole.

5. A method of treating a vaginal fungal infection comprising oral administration of fluconazole in an amount of from about 75 to about 150 mg/day for one to three days and concurrent vulvar administration of an external topical composition comprising an antifungal agent selected from the group consisting of miconazole nitrate, fluconazole, terconazole, butoconazole, clotrimazole, and combinations thereof.

6. A method of treating a vaginal fungal infection comprising oral administration of fluconazole in an amount of from about 75 to about 150 mg/day and concurrent vulvar administration of an external topical composition comprising an anti-inflammatory agent.

7. A method of treating a vaginal fungal infection comprising oral administration of fluconazole in an amount of from about 125 to about 150 mg and concurrent vulvar administration of an external topical composition comprising an emollient agent.

8. A method of treating a vaginal bacterial infection comprising oral administration of metronidazole in an amount of from about 500 single oral dose and concurrent vulvar administration of an external topical composition comprising 0.75% metronidazole gel.

9. A method of treating a local viral infection comprising oral administration of acyclovir (Zovirax), famcyclovir (Famvir), valcyclovir (Valtrex) in a daily dosage of from about 250 mg to about 2000 mg/day and concurrent local administration of an external topical composition comprising emollient or local anesthetic ingredients.

10. A kit comprising an oral dosage form of fluconazole and an external topical composition.

11. An applicator containing an external topical composition comprising stick, wipe, sanitary pad, pantyliner, wash, spray and roll-on.

12. A method of treating a topical infection comprising oral administration of an effective amount of anti-infective agent and concurrent local administration of an external topical composition comprising an effective amount of a topical relief agent to ameliorate local symptoms.

13. A method according to claim 12 wherein said oral administration comprises administering a composition comprising fluconazole and said topical composition comprises miconazole nitrate.

14. A method of treating onychomycosis comprising oral administration of an effective amount of anti-fungal agent and concurrent local administration of an external composition to a nail affected by onychomycosis comprising an effective amount of an anti-fungal agent to ameliorate local symptoms.

15. A method of treating a local bacterial infection comprising oral administration of an effective amount of anti-infective agent and concurrent local administration of an external topical composition comprising an effective amount of a topical relief agent to ameliorate local symptoms.

16. A method according to claim 14 wherein said anti-fungal agent is fluconazole.
